(19) United States
(12) Patent Application Publication
(10) Pub. No.: US 2014/0234445 A1
(43) Pub. Date: Aug. 21, 2014

(54) Composition comprising an extract of combined herbs consisting of ginseng and Vitis genus plant for preventing and treating Neuro-degenerative disease and enhancing memory power

(71) Applicant: GINSENG SCIENCE INC., Seoul (KR)

(72) Inventors: Bok Deuk Kim, Seoul (KR); Jeong Hill Park, Seoul (KR); Jonghoon Ryu, Seoul (KR)

(21) Appl. No.: 14/347,010

(22) PCT Filed: Nov. 5, 2012
(86) PCT No.: PCT/KR2012/009226

§ 371 Date: Mar. 25, 2014

(30) Foreign Application Priority Data
Dec. 14, 2011 (KR) 10-2011-0134113

(51) Int. Cl. A61K 36/87; A23L 2/52; A23L 1/30; A61K 36/258

(52) U.S. Cl. CPC A61K 36/87; A61K 36/258; A23L 2/52; A23L 1/3002
USPC 424/728

(57) ABSTRACT

The present invention relates to compositions comprising an extract of combined herbs consisting of ginseng and Vitis genus plant for treating and preventing neuro-degenerative disease and enhancing memory power. The combined inventive extract showed synergistic enhancing effect through passive avoidance test using by scopolamine-induced memory injured animal model comparing with respective extract, therefore the inventive extract can be useful in treating or preventing neuro-degenerative disease.

### BACKGROUND OF THE INVENTION

1. Technical Field

The present invention relates to a pharmaceutical composition and health functional food comprising an extract of combined herbs consisting of ginseng and Vitis genus plant for treating and preventing neuro-degenerative disease and enhancing memory power and the use thereby.

2. Background Art

CNS (Central Nervous System) consisting of brain and spinal cord which plays a main role in regulating life phenomenon is a essential organ governing all the human function through from sensory and (in) voluntary movement to thinking, memory, motion, language etc. Accordingly, a rapidly progressed apoptosis of neuronal cell caused by stroke, trauma etc as well as slowly progressed apoptosis such as degenerative disease occurring in CNS caused by senile dementia for example, Alzheimer's disease or Parkinson disease etc result in irreversible functional disorder of neuronal network, which give rise to immortal failure of human function in the end. Among them, the patients suffering from Alzheimer disease, a representative senile dementia have been increased in proportion to both of extended life-span and modernized welfare facility. According to the public survey of Korea Institute for Health and Social Affair, the ratio of older people among Korean people exceeds 7% in 2000, reaches to 8.3% (3,970,000) and shall approach to 14.4% in 2019. Especially, the ratio of more than 65 years old patient suffering with senile dementia is presumed to 8.2% in Korea. In Western countries, about 10% among more than 65 years old and about 40-50% among 80 years old patient suffers with senile dementia. Since more than five million patients suffer with the disease, the medical expense caused thereby is presumed to hundred billion dollars in a year. There have been found that more than about two hundred thousand people are suffering from dementia in Korea. In America, it has been presumed the number of the patients be increased to two fold than the number of present patients in 2030 and fourteen million (more than 350%) in 2050.

Since Alzheimer's disease initiated with cognitive function disorder is one of long-term degenerative diseases resulting in the breakdown of human nature, there have been tried to develop effective and preventive drugs till now, for example, acetylcholineesterase inhibitor such as Aricept® (Pfizer Co.), Exelon® (Novartis Co.), Reiminyl® (Janssen Co.) or NMDA receptor antagonist such as Ebixa® (Lundbeck Co.). However, the acetylcholine esterase inhibitor could just alleviate reduced cognitive ability and could not satisfactorily treat etiological cause of the disease. Although the drug shows temporarily alleviated effect on only some of patients (about 40-50%), it could not maintain it's potency for a long time moreover it shows various adverse response such as hepato-toxicity, vomiting, anorexia in case of long-term treatment. Accordingly, there has been urgently needed to develop new therapeutic agent to prevent and treat the disease nowadays. Many multi-national pharmaceutical companies have been invested on the development in a large scale and in particular, focused in the development for beta- or gamma secretase inhibitor reducing the reproduced amount of beta-amyloid consisting of about 40 amino acids which has been presumed to be an etiological factor of Alzheimer disease. The basic study on the Alzheimer disease has been actively attempted in Korea however the development of Alzheimer treating agent has been merely progressed till now. Since there have been found in animal model test as well as clinical trial that the development of gamma secretase inhibitor is associated with considerable toxicity, it has been proved to be not recommendable whereas the development of beta secretase inhibitor is recommendable as proven by gene deficiency transformed animal model test. It is also regarded as a safe tool to focus on targeting the factors involved in beta amyloid aggregation. There has been reported that ‘phenserine’ developed by Axonyx Co. in USA has been progressed in Clinical trial 2 phase and it shows dual activities of inhibiting cholinesterase as well as beta amyloid aggregation. (Greig et al., J. Med. Chem. 44 pp 4062-4071, 2001; www.medicalnewstoday.com; www.alzforum.org/drg/drc)

The development of vaccine using beta amyloid has been known as another possible method. There has been reported that the serial study on the vaccine progressed by Elan Co. failed because of its un-predictable adverse response such as encephalitis during clinical trial. However, it has been reported that beta amyloid vaccine could alleviate cognitive function in animal model test and improve the activity of brain cell as well as damaged brain neuronal cells, resulting in alleviating Alzheimer syndrome. (Janus et al., Nature 408, pp 979-982, 2000; Morgan et al., Nature 408, pp 982-985, 2000)

It is known that there are many genus of Panax genus plants belonged to Araliaceae, for example, Panax ginseng distributed or cultivated in far-eastern Asia region, Panax quinquefolia in America and Canada, Panax Notoginseng in China, Panax trifolia in eastern region of north America, Panax vietnamensis in Vietnam, Panax elegatior, Panax wangianus and Panax bipinratifidus etc.

Recently, there have been several attempts to strengthen pharmacological effects among ginseng by modifying the method of ginseng processing, for example, Park et al developed new methods for preparing a processed ginseng under specific high temperature and high pressure as disclosed in Korean Patent Registration No. 192678 and U.S. Pat. No. 5,776,460, which changes main ginseng saponins such as ginsenosides Rb1, Rb2, Rc and Rd, into new saponins such as ginsenosides Rg3, Rg5, Rk1, Rk2, Rk3, Rs1, Rs2, Rs3, F4, Rh2, Rh4 and compound K showing new and more potent pharmacological effects, for examples, anti-oxidative activity, anti-cancer activity and alleviating activity of blood circulation etc (Kim W Y et al., J. Nat. Prod., 63(12), pp 1702-1704; Kwon S H et al., J. Chromatogr. A., 921(2), pp 335-339, 2001). Especially, ginsenosides Rg3, Rg5 and Rk1 has been known to show most potent pharmacological activities among them, for example, neuro-protective activity, anti-dementia activity, memory-enhancing activity etc (Yang L L et al., J. Pharm. Pharmacol., 61, pp 375-380, 2009; Bao H. Y., et al., Arch. Pharm. Res., 28(3), pp 335-342, 2005). Accordingly, these new ginsenosides can be produced in the root, stem or leaf of any Panax genus plants such as Panax ginseng, Panax quinquefolia, Panax notoginseng, Panax japonica, Panax trifolia, Panax pseudoginseng, Panax vietnamensis, Panax elegatior, Panax wangianus and Panax bipinratifidus which contains dammarane glycoside through the processing method of Park et al (Korean Patent Registration No. 192678 and U.S. Pat. No. 5,776,460).

It is known that there are many genus of Vitis genus plants belonged to Vitaceae, for example, Vitis vinifera, Vitis amurensis, Vitis flexuosa, Vitis coignetiae, Vitis thunbergii, Vitis riparia, and Vitis kaempferi etc, and the extract of plant shows various pharmacological activities such as anti-dementia activity, memory-enhancing activity etc (Jeong H Y et al., Arch. Pharm. Res., 33, pp 1655-1664, 2010; Arzi A et al., Toxicology Letters, 180, pp. S126, 2008).

Resveratrol and the analogues have been reported to be contained in the extract of Vitis genus plants. Especially, vitisin A and gamma-viniferin among them have been known to show potent inhibitory effect on acetylcholine esterase enzyme and vitisin A, vitisin B, viniferin, ampelopsin A and the mixture thereof also show potent inhibitory effect on BACE-1 (beta-site APP-cleaving enzyme 1) enzyme (Korea Patent Publication No. 10-2004-0069762; Jang M H et al., Phytother. Res., 22(4), pp 544-549, 2008; Jang M H et al., Biol. Pharm., Bull., 30, pp 1130-1134, 2007).

However, there has been not reported or disclosed about the synergistic effect of the extract of combined herbs consisting of ginseng and Vitis genus plant for treating and preventing neuro-degenerative disease and enhancing memory power in any of the above cited literatures, the disclosures of which are incorporated herein by reference.

To investigate the synergistic effect on the neuro-degenerative disease of novel combination of ginseng and Vitis genus plant, the inventors of the present invention have studied tested biological activity using passive avoidance test, and finally completed present invention by confirming that the novel combination showed stronger synergistic memory enhancing effect than sole plant extract.

### SUMMARY OF THE INVENTION

### Disclosure of Invention

Accordingly, the present invention provides a pharmaceutical composition comprising an extract of combined herbs consisting of ginseng and Vitis genus plant for treating and preventing neuro-degenerative disease and enhancing memory power.

The term “ginseng” disclosed herein comprises a root, leaf, fruit, or rhizome of a processed ginseng such as red ginseng or, a processed ginseng produced by heating at the temperature ranging from 70 to 200° C., preferably, 80 to 180° C. for the period ranging from 0.5 to 20 hours, preferably, 2 to 16 hours so as to make a ratio of ginsenoside (Rg3+Rg5+Rk1) to (Rb1+Rb2+Rc+Rd) of over 1.0; or a wild ginseng such as white ginseng, of which ginseng is selected from Panax ginseng, Panax quinquefolia, Panax notoginseng, Panax japonica, Panax trifolia, Panax pseudoginseng, Panax vietnamensis, Panax elegatior, Panax wangianus or Panax bipinratifidus, preferably, Panax ginseng

The term “ginsenoside Rg3” disclosed herein includes two isomers of ginsenoside (20-S) and (20-R).

The term “Vitis genus plant” disclosed herein comprises a fruit, seed, root, leaf or stem, preferably, root, stem or fruit of Vitis vinifera, Vitis amurensis, Vitis flexuosa, Vitis coignetiae, Vitis thunbergii, Vitis riparia, and Vitis kaempferi etc belongs to Vitaceae family, preferably, Vitis vinifera or Vitis amurensis in the present invention.

The term “extract” disclosed herein is soluble in water, C₁-C₄ lower alcohol, or the mixtures thereof, preferably water or 10-90% ethanol in water, more preferably, 50-80% ethanol in water.

The extract disclosed herein comprise the extract of combined herbs consisting of ginseng and Vitis genus plant with the mixed ratio ranging from 1:0.01-1000 (w/w), preferably, 1: 1-10 (w/w), more preferably, 1: 1-5 (w/w) in the present invention.

The term “neuro-degenerative disease” disclosed herein comprises stroke, Alzheimer type dementia, cerebrovascular type dementia, Huntington's disease, Pick's disease, Creutzfeldt-jakob's disease, dementia caused by cephalic damage, Parkinson's disease, and the like, preferably, Alzheimer type dementia, cerebrovascular type dementia or Parkinson's disease.

Accordingly, it is an object of the present invention to provide a pharmaceutical composition comprising an extract of combined herbs consisting of ginseng and Vitis genus plant, as an active ingredient in an amount effective to prevent and treat neuro-degenerative disease, together with a pharmaceutically acceptable carrier or diluents.

It is another object of the present invention to provide a use of an extract of combined herbs consisting of ginseng and Vitis genus plant for manufacture of medicament employed for treating or preventing neuro-degenerative disease in human or mammal.

It is the other object of the present invention to provide a method for treating neuro-degenerative disease in a mammal or animal suffering from said disease comprising administering an effective amount of an extract of combined herbs consisting of ginseng and Vitis genus plant, together with a pharmaceutically acceptable carrier to said mammal or animal in need thereof

The herbs, which can be used in the present invention, include the same genus plants which would be apparent to those skilled in the art and have been used for identical or similar purpose and can be substituted for the prevention and treatment of the diseases.

Hereinafter, the present invention is described in detail.

For the preparation of ginseng of the present invention, for example, the dried fruit, seed, root, leaf or stem of each plant materials, i.e., ginseng and Vitis genus plant was cut into small pieces, especially, the ginseng is heated with high temperature ranging from 70 to 200° C., preferably, 80 to 180° C. for the period ranging from 0.5 to 20 hours, preferably, 2 to 16 hours so as to make a ratio of ginsenoside (Rg3+Rg5+Rk1) to (Rb1+Rb2+Rc+Rd) of over 1.0; the plant materials mixed with 1 to 50-fold, preferably, 5 to 15-fold weight (w/v) of water, C₁-C₄ lower alcohol, such as methanol, ethanol, butanol or the mixtures thereof, preferably water or 10-90% ethanol in water, more preferably, 50-80% ethanol in water and extracted by reflux extraction, cold water extraction, ultra-sonication or other conventional extraction, preferably by reflux extraction with the temperature ranging from 10 to 150° C., preferably, 50 to 100° C. for the period ranging from 0.5 to 20 hours, preferably, 2 to 16 hours; the residue was filtered or precipitated with cold ethanol to suspended solution prepared by adding water; the filtrate was dried at the temperature ranging from 40 to 80° C., preferably from 50 to 70° C.,; and each dried extract is mixed with the mixed ratio based on the dried weight of each herb (w/w) ranging from 1:0.01-1000, preferably, 1: 1-10, more preferably, 1: 1-5 to obtain inventive combined extract of the present invention.

Accordingly, the present invention also provides a method for preparing extract of combined herbs consisting of ginseng and Vitis genus plant comprising the steps of; drying the fruit, seed, root, leaf or stem of ginseng and Vitis genus plant and heating the ginseng with high temperature ranging from 70 to 200° C. for the period ranging from 0.5 to 20 hours so as to make a ratio of ginsenoside (Rg3+Rg5+Rk1) to (Rb1+Rb2+Rc+Rd) of over 1.0 at 1^{st} step; mixing the plant materials with 1 to 50-fold weight (w/v) of water, C₁-C₄ lower alcohol or the mixture thereof and extracting the solution by reflux extraction, cold water extraction, ultra-sonication or other conventional extraction at the temperature ranging from 10 to 150° C. for the period ranging from 0.5 to 20 hours; filtering the residue or precipitating the suspended solution prepared by adding water with cold ethanol to afford their supernatant at 2^{nd} step; drying the filtrate or the supernatant at the temperature ranging from 40 to 80° C.; mixing each dried extract with the mixed ratio based on the dried weight of each herb (w/w) ranging from 1:0.01-1000 to obtain inventive combined extract of the present invention.

The combined inventive extract prepared by the above-described method shows synergistic enhancing effect on passive avoidance test using by scopolamine-induced memory injured animal model comparing with respective extract, therefore the inventive extract can be useful in treating or preventing neuro-degenerative disease.

Accordingly, it is another object of the present invention to provide the pharmaceutical composition comprising an extract of combined herbs consisting of ginseng and Vitis genus plant prepared by the above-described method, as an active ingredient in an amount effective to prevent and treat neuro-degenerative disease, together with a pharmaceutically acceptable carrier or diluents.

In accordance with the other aspect of the present invention, there is also provided a use of an extract of combined herbs consisting of ginseng and Vitis genus plant prepared by the above-described method for manufacture of medicines employed for treating or preventing neuro-degenerative disease in mammals including human as an active ingredient in amount effective to treat or prevent neuro-degenerative disease.

In accordance with the other aspect of the present invention, there is also provided a method of treating or preventing neuro-degenerative disease in a mammal comprising administering to said mammal an effective amount of an extract of combined herbs consisting of ginseng and Vitis genus plant prepared by the above-described method, together with a pharmaceutically acceptable carrier thereof into the mammals including human suffering from said disease.

Inventive composition of the present invention has no toxicity and adverse effect therefore can be used with safe.

In terms of pharmaceutical composition of the present invention, the inventive extract preferably should be present between 0.01 to 99%, and more preferably between 0.02 to 50%. The above composition does not limit the invention in no way.

Therefore, the present invention may be prepared by mixing the inventive extract with the pharmaceutically acceptable carriers, adjuvants or diluents as pharmaceutical composition for prevention and treatment of purposed disease or disorders.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starch, acacia rubber, alginate, gelatin, calcium phosphate, calcium silicate, cellulose, methyl cellulose, polyvinyl pyrrlidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like.

The desirable dose of the inventive extract varies depending on the condition and the weight of the subject, severity, drug form, route and period of administration, and may be chosen by those skilled in the art. However, in order to obtain desirable effects, it is generally recommended to administer at the amount ranging from 0.0001 to 100 mg/kg, preferably 0.001 to 100 mg/kg by weight/day of the inventive compounds of the present invention. The dose may be administered in single or divided into several times per day.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intrathecal, epidural or intracerebroventricular injection.

The present invention provides a health functional food comprising an extract of combined herbs consisting of ginseng and Vitis genus plant as an active ingredient for alleviating or preventing neuro-degenerative disease and enhancing memory power.

The present invention also provides a health functional food comprising an extract of combined herbs consisting of ginseng and Vitis genus plant, and a sitologically acceptable additive for alleviating or preventing neuro-degenerative disease and enhancing memory power.

In a preferred embodiment, it is the other object of the present invention to provide a health functional food or health care food comprising an extract of combined herbs consisting of ginseng and Vitis genus plant for alleviating or preventing neuro-degenerative disease and enhancing memory power, together with a sitologically acceptable additive.

The present invention provides a food additive comprising an extract of combined herbs consisting of ginseng and Vitis genus plant as an active ingredient for alleviating or preventing neuro-degenerative disease and enhancing memory power.

The crude drug composition of inventive health functional food or health care food is used in the form of pulverized form thereof, extracted form therefrom or dried extract form thereof.

The term “a sitologically acceptable additive” disclosed herewith comprises the additive which can be conventionally available well-known in the art, for example food additive lists published on U.S. Food and Drug Administration (See, www.fda.gov/food).

The health functional food composition for preventing and improving purposed diseases could contain about 0.01 to 95 w/w %, preferably 0.5 to 80 w/w % of the above crude extract based on the total weight of the composition.

Above described the crude drug composition therein can be added to food, additive or beverage for prevention and improvement of purposed diseases. For the purpose of preventing and improving purposed diseases, wherein, the amount of above described crude drug composition in food or beverage may generally range from about 0.1 to 15 w/w %, preferably 1 to 10 w/w % of total weight of food for the health food composition and 1 to 30 g, preferably 3 to 10 g on the ratio of 100 ml of the health beverage composition.

Providing that the health beverage composition of present invention contains above described extract as an essential component in the indicated ratio, there is no particular limitation on the other liquid component wherein the other component can be various deodorant or natural carbohydrate etc such as conventional beverage. Examples of aforementioned natural carbohydrate are monosaccharide such as glucose, fructose etc; disaccharide such as maltose, sucrose et al.; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartame etc, may be useful favorably. The amount of above described natural carbohydrate generally ranges from about 1 to 20 g, preferably 5 to 12 g in the ratio of 100 ml of present beverage composition.

The other components than aforementioned composition are various nutrients, a vitamin, a mineral or and electrolyte, synthetic flavoring agent, a coloring agent and improving agent in case of cheese, chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition

### Advantageous Effects

In accordance, the present invention provides a use of extract of processed Panax genus plant so as to make a ratio of ginsenoside (Rg3+Rg5+Rk1) to (Rb1+Rb2+Rc+Rd) of over 1.0 as an active ingredient in an effective amount to prevent and treat neuro-degenerative disease and to enhance memory.

### BEST MODE FOR CARRYING OUT THE INVENTION

It will be apparent to those skilled in the art that various modifications and variations can be made in the compositions, use and preparations of the present invention without departing from the spirit or scope of the invention.

### MODE FOR THE INVENTION

The present invention is more specifically explained by the following examples. However, it should be understood that the present invention is not limited to these examples in any manner.

### Comparative Example 1

### Preparation of the Extract of Ginseng

1.1. Extract of Ginseng (PG)

1 kg of Panax ginseng root was mixed with 4 L of mixture solvent of ethanol and water (60:40) and refluxed for 4 hrs in water bath repeatedly. The extract was filtered and dried to afford 250 g of the extract of ginseng (designated as “PG” hereinafter).

1.2. Extract of Processed Ginseng (HPG)

Processed ginseng was prepared in accordance with the procedure disclosed in the literature (Kim W Y et al., J. Nat. Prod., 63(12), pp 1702-1704; Kwon S W et al., J. Chromatogr A., 921(2), pp 335-339, 2001). 1 kg of dried plant material of Panax genus was cut into small pieces and the sliced pieces were heated at 110° C. for 3 hours in autoclave (JEIO TECH., AC-11, Korea). The processed ginseng was mixed with 4 L of mixture solvent of ethanol and water (50:50) and heated for 4 hours by reflux extraction in water bath repeatedly. The residue was filtered and then the filtrate was evaporated to obtain 300 g of the extract of processed ginseng (designated as “HPG” hereinafter).

### Comparative Example 2

### Preparation of the Extract of Vitis Genus Plant

1.1. Fruit Extract of Vitis Genus Plant (VV-F/VA-F)

1 kg of each fruit of Vitis vinifera and Vitis amurensis was mixed with 4 L of mixture solvent of ethanol and water (60:40) and refluxed for 4 hrs in water bath repeatedly. The extract was suspended in 200 ml of water and 800 ml of anhydrous ethanol was added thereto to precipitate. The supernatant was collected and dried to afford 50 g and 60 g of the fruit extract of Vitis vinifera and Vitis amurensis respectively (designated as “VV-F and VA-F” respectively, hereinafter).

1.2. Stem Extract of Vitis Genus Plant (VV-S/VA-S)

400 g of each stem of Vitis vinifera and Vitis amurensis was mixed with 4 L of mixture solvent of ethanol and water (60:40) and refluxed for 4 hrs in water bath repeatedly. The extract was filtered and concentrated under vaccuo to afford 30 g and 35 g of the stem extract of Vitis vinifera and Vitis amurensis respectively (designated as “VV-S and VA-S” respectively, hereinafter).

1.3. Root Extract of Vitis Genus Plant (VV-R/VA-R)

400 g of each root of Vitis vinifera and Vitis amurensis was mixed with 4 L of mixture solvent of ethanol and water (60:40) and refluxed for 4 hrs in water bath repeatedly. The extract was filtered and concentrated under vaccuo to afford 40 g and 45 g of the stem extract of Vitis vinifera and Vitis amurensis respectively (designated as “VV-R and VA-R” respectively, hereinafter).

### Example 1

### Preparation of the Combined Extract of Ginseng and Vitis Genus Plant

The extract prepared in Comparative Examples was mixed with the various mixed ratio (w/w) and used in following Experiments.

### Reference Example 1

### Preparation of Experiment

### 1.1. Reagent and Drugs.

Scopolamine and Donepezil® were procured from Sigma-Aldrich Chemistry Co. and the other reagents were commercially available in the market or company.

### 1.2. Experimental Animals

6 weeks old female ICR mice weighing 26-28 g (Orient Bio Co. Ltd, www.orientbio.co.kr, Korea) were bred and were used for experiment after five days adaptation. The room was controlled by automatic light system from 7:00 A.M. to 7:00 P.M, for 12 hours, and the temperature was adjusted to 23±2° C., humidity was adjusted to 50±10° C.%. The animal feed and tap water were fed freely.

### 1.3. Static Analysis

The statistical significance of all results was tested by ANOVA (one way analysis of variance) test and the significance was verified with Student-Newman-Keuls-test (p<0.05).

### Experimental Example 1

### The Effect of Sole Treatment

In order to investigate the effect of sole treatment with each extract prepared in Comparative Examples, following passive avoidance task using by scopolamine-induced memory impaired animal was performed according to the modified method disclosed in the literature (Kim et al., Prog. Neuropsychopharmacol., Biol. Psychiatry, 34, pp 1011-1017, 2010).

### 1-1. Sample Treatment

The extract prepared in Comparative Examples were dissolved in 10% Tween 80 (Polyoxyethylene sorbitan monooleate: Sigma, USA) solution and the solution was orally administrated into the animals. Various concentrations of the extract of ginseng (10, 20, 5 and 2.5 mg/kg) and Vitis genus plant (200, 100, 50 and 25 mg/kg) were used as test groups of which group consists of 10 mice and the positive control group treated with 5 mg/kg of Donepezil and negative control group treated with only 10% Tween 80 were used in the following experiments.

### 1-2. Passive Avoidance Test

Black avoidance shuttle box was divided into two chambers of equal size (18 cm L×9.5 cm W×17 cm H) partitioned by compartment door (4 cm L×3.5 cm W) allowing electricity to run on the floor of the dark compartment. A light chamber is equipped with a 20-W lamp on the hinged plexiglass lid and the mice were allowed to enter dark chamber through compartment door.

30 mins after the drug administration, scopolamine (Sco) was intraperitoneally administrated into the mice at the dose of 1 mg/kg. 30 mins after the scopolamine administration, the mice were initially placed in the light chamber and allowed for habituation. The door was then opened and as soon as mice preferring darkness went out from light chamber and entered the dark chamber the door was closed immediately, and an electric shock (0.5 mA, 3 s, once) was delivered to the mouse through the grid floor for 3 sec in the training session.

The experiments consisted of training and test sessions. At 24 hours after the training session, the identical experiment was performed again with mouse to measure the latency time staying at the light chamber. The data was regarded as the index which meant the memory on previous training by 0.25 mA of electronic shock for 3 second. Latency to enter the dark compartment from the light compartment was measured as a step through latency. If it did not enter the dark chamber within the cut-off time (300 sec), it was assigned a value of 300 sec as its latency.

As shown in Table 1 and 2, the change of latency time means the decline or recovery of memory and the lengthened latency time means the increased memory. In the sham operated control group, there was no change in latency time and in the negative control group administered with solvent. 20 mg/kg treatment group with HPG and 200 mg/kg treatment group with VVR showed significantly improved memory however 200 mg/kg treatment group with sole VVS, VA-S and VA-FR did not show significant memory enhancing effect.

TABLE 1
Memory improving effect of sole sample treatment (HPG)

| Group | Latency time (sec) |
|---|---|
| Negative Control | 239 ± 22 |
| Scopolamine treatment (Sco) | 43 ± 7* |
| Scopolamine + 2.5 mg/kg of HPG | 33 ± 5 |
| Scopolamine + 5 mg/kg of HPG | 53 ± 19 |
| Scopolamine + 10 mg/kg of HPG | 122 ± 33 |
| Scopolamine + 20 mg/kg of HPG | 157 ± 31^{#} |
| Scopolamine + Donepezil | 146 ± 33^{#} |

*P < 0.05, comparison with negative control;
^{#}P < 0.05, comparison with scopolamine treatment group

TABLE 2
Memory improving effect of sole sample
treatment (Vitis genus plant)

| | Latency Time (sec) | | | |
|---|---|---|---|---|
| Sample | VV-F | VV-R | VA-S | VA-F |
| Negative Control | 232 ± 20 | 270 ± 16 | 248 ± 19 | 248 ± 21 |
| Scopolamine | 46 ± 10* | 41 ± 10* | 44 ± 8* | 44 ± 7* |
| treatment (Sco) | | | | |
| Sco + sample | 64 ± 15 | 63 ± 15 | 82 ± 21 | 52 ± 10 |
| (25 mg/kg) | | | | |
| Sco + sample | 78 ± 14 | 67 ± 11 | 85 ± 20 | 68 ± 16 |
| (50 mg/kg) | | | | |
| Sco + sample | 88 ± 18 | 92 ± 27 | 79 ± 23 | 92 ± 18 |
| (100 mg/kg) | | | | |
| Sco + sample | 121 ± 24 | 168 ± 33^{#} | 42 ± 4 | 80 ± 14 |
| (200 mg/kg) | | | | |
| Scopolamine + | 153 ± 30^{#} | 161 ± 30^{#} | 143 ± 24^{#} | 141 ± 25^{#} |
| Donepezil | | | | |

*P < 0.05, comparison with negative control;
^{#}P < 0.05, comparison with scopolamine treatment group

### Experimental Example 2

### The Effect of Combined Treatment

In order to investigate the synergistic effect of combined treatment with the extract prepared in Examples, following passive avoidance task using by scopolamine-induced memory impaired animal was performed according to the modified method disclosed in the literature (Kim et al., Prog. Neuropsychopharmacol., Biol. Psychiatry, 34, pp 1011-1017, 2010).

### 1-3. Sample Treatment

Each extract prepared in Comparative Example 1 (HPG) and 2 (Vitis genus plant) was combined with the different sorts, i.e., (a) HPG+VV-S, (b) HPG+VV-R, (c) HPG-VA-S and (d) HPG-VA-F, with the different mixed ratios, i.e., (a) 1:1 (v/v), (b) 1:2 (v/v), and (c) 1:3 (v/v)), in order to confirm the synergistic effect, i.e., the final dose of combined sample was diluted to ¾ of the effective dose of sole sample (1; ½ dose of HPG+½ dose of vitis genus plant, etc. For example, the combined sample mixed with the ratio of 1:1 was prepared by mixing HPG (20 mg/kg×½×¾) with the extract of Vitis genus plant (each effective dose of sole sample×½×¾). Scopolamine dissolved in physiological saline solution was intraperitoneally administrated at the dose of 1 mg/kg and the positive control group treated with 5 mg/kg of Donepezil and negative control group treated with only 10% Tween 80 were used in the following experiments.

### 1-4. Passive Avoidance Test

Black avoidance shuttle box was divided into two chambers of equal size (18 cm L×9.5 cm W×17 cm H) partitioned by compartment door (4 cm L×3.5 cm W) allowing electricity to run on the floor of the dark compartment. A light chamber is equipped with a 20-W lamp on the hinged plexiglass lid and the mice were allowed to enter dark chamber through compartment door.

30 mins after the drug administration, scopolamine (Sco) was intraperitoneally administrated into the mice at the dose of 1 mg/kg. 30 mins after the scopolamine administration, the mice were initially placed in the light chamber and allowed for habituation. The door was then opened and as soon as mice preferring darkness went out from light chamber and entered the dark chamber the door was closed immediately, and an electric shock (0.5 mA, 3 s, once) was delivered to the mouse through the grid floor for 3 sec in the training session.

The experiments consisted of training and test sessions. At 24 hours after the training session, the identical experiment was performed again with mouse to measure the latency time staying at the light chamber. The data was regarded as the index which meant the memory on previous training by 0.25 mA of electronic shock for 3 second. Latency to enter the dark compartment from the light compartment was measured as a step through latency. If it did not enter the dark chamber within the cut-off time (300 sec), it was assigned a value of 300 sec as its latency.

As shown in Table 3, the change of latency time means the decline or recovery of memory and the lengthened latency time means the increased memory. It has been confirmed that there showed significant reduce in scopolamine treatment group comparing with control group. All the combined group with (1) HPG and VV-F (HPP+VV-F, 1:2), (2) HPG and VV-R (HPP+VV-R, 1:1, 1:2, and 1:3) and (3) HPG and VA-S(HPP+VA-S, 1:3) showed synergistic effect on memory improved effect comparing with sole treatment group.

TABLE 3
Memory improving effect of combined sample treatment

| | Latency Time (sec) | | | |
|---|---|---|---|---|
| | HPG + | HPG + | HPG + | HPG + |
| | VV-F | VV-R | VA-S | VA-F |
| Sample | (1:2, v/v) | (1:2, v/v) | (1:3, v/v) | (1:1, v/v) |
| Negative Control | 248 ± 15 | 273 ± 13 | 249 ± 15 | 253 ± 15 |
| Scopolamine | 51 ± 9* | 42 ± 9* | 32 ± 8* | 46 ± 9* |
| treatment (Sco) | | | | |
| Sco + combined | 130 ± 31^{#} | 140 ± 24^{#} | 104 ± 21^{#} | 98 ± 30^{#} |
| sample | | | | |
| Scopolamine + | 152 ± 27^{#} | 157 ± 24^{#} | 135 ± 23^{#} | 154 ± 27^{#} |
| Donepezil | | | | |

*P < 0.05, comparison with negative control;
^{#}P < 0.05, comparison with scopolamine treatment group

### Example 3

### Analysis of the Gensenoside Amount of Processed Ginseng Product

In order to compare the component analysis between the extract of natural ginseng and processed ginseng, following HPLC analysis was performed according to the method disclosed in the literature (Kwon S. W. et al., J. Chromatogr., A, 921(2), pp. 335-339, 2001).

1 g of each sample prepared in Comparative Examples was dissolved in 20 ml methanol to use as a sample.

At the result, it was confirmed that the sum of (Rg3+Rg5+Rk1) is higher than the sum of (Rb1+Rb2+Rc+Rd) through the analysis of each relative peak area of ginsenosides in HPG, differently from the result of natural ginseng extract (PG) (See Table 4)

TABLE 4

| | Rb1 + Rb2 + Rc + Rd | Rg3 + Rg5 + Rk1 | |
|---|---|---|---|
| | (A, %) | (B, %) | B/A |
| PG | 18.8 | — | — |
| HPG | 4.5 | 10.2 | 2.27 |

### Example 4

### Acute Toxicity Test of Oral Administration

Acute toxicity test was performed by using four-weeks-old ICR mouse (Orientbio, Japan) according to method disclosed in the literature (Haschek W M and Rousseaux C G, Handbook of toxicologic pathology, Published by Academic press, Inc., New York, pp 293-295, 1991).

The inventive combined extract of the present invention dissolved in water was administrated orally to each group consisting of 5 mice in a dose of 2 g/kg/10 ml. After administration, the mortality of the mice and clinical symptom, body weight change was observed and hematologic test and hematological biochemistry test was performed. Whether abdominal organ and thoracic organ is abnormal was observed with the naked eye by an autopsy.

As the result, there was no toxicity effect on clinical symptom, mortality, body weight change and gross finding in all the animals. Accordingly these results suggested that the mixed herbal extract of the present invention were potent and safe substance with over 5000 mg/kg of the minimum lethal dose of oral administration.

Hereinafter, the formulating methods and kinds of excipients of pharmaceutical compositions or health functional food will be described, but the present invention is not limited to them.

### Preparation of Powder

| | | |
|---|---|---|
| HPG + VV-F (1:2) | 500 | mg |
| Lactose | 100 | mg |
| Talc | 10 | mg |

Powder preparation was prepared by mixing above components and filling sealed package.

### Preparation of Tablet

| | | |
|---|---|---|
| HPG + VV-R (1:10) | 500 | mg |
| Corn Starch | 100 | mg |
| Lactose | 100 | mg |
| Magnesium Stearate | 2 | mg |

Tablet preparation was prepared by mixing above components and entabletting.

### Preparation of Capsule

| | | |
|---|---|---|
| HPG + VA-S (1:5) | 500 | mg |
| Crystallized cellulose | 3 | mg |
| Lactose | 14.8 | mg |
| Magnesium Stearate | 0.2 | mg |

Capsule preparation was prepared by mixing above components and filling gelatin capsule by conventional gelatin preparation method.

### Preparation of Injection

| | | |
|---|---|---|
| HPG + VA-F (1:2) | 100 | mg |
| Mannitol | 180 | mg |
| Distilled water for injection | 2974 | mg |
| Na₂HPO₄•12H₂O | 26 | mg |

Injection preparation was prepared by dissolving active component and then filling all the components in 2 ml ample and sterilizing by conventional injection preparation method.

### Preparation of Liquid

| | | |
|---|---|---|
| HPG + VV-F (1:10) | 1000 | mg |
| Isomerized sugar | 10 | g |
| Mannitol | 5 | g |

| | |
|---|---|
| Distilled water | optimum amount |

Liquid medicine was prepared by dissolving the components to distilled water with a proper dose of lemon scent, mixing, adjusting to 100 ml with distilled water in brown bottle and sterilizing by conventional liquid medicine preparation method.

### Preparation of Health Functional Food

| HPG + VA-F (1:2) | 500 | mg |
|---|---|---|
| Vitamin mixture | optimum amount | |
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Amide nicotinic acid | 1.7 | mg |
| Folic acid | 50 | μg |
| Calcium pantothenic acid | 0.5 | mg |
| Mineral mixture | optimum amount | |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 100 | mg |
| Magnesium chloride | 24.8 | mg |

The above-mentioned vitamin and mineral mixture may be varied in many ways. Such variations are not to be regarded as a departure from the spirit and scope of the present invention.

### Preparation of Health Beverage

| | | |
|---|---|---|
| HPG + VV-R (1:10) | 1000 | mg |
| Citric acid | 1000 | mg |
| Oligosaccharide | 100 | g |
| Apricot concentration | 2 | g |
| Taurine | 1 | g |
| Distilled water | 900 | ml |

Health beverage preparation was prepared by dissolving active component, mixing, stirring at 85° C. for 1 hour, filtering and then filling all the components in 2 l container and sterilizing by conventional health beverage preparation method.

The invention being thus described, may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the combined inventive extract prepared by the above-described method shows synergistic enhancing effect through passive avoidance test using by scopolamine-induced memory injured animal model comparing with respective extract, therefore the inventive extract can be useful in treating or preventing neuro-degenerative disease.

1. A pharmaceutical composition comprising an extract of combined herbs consisting of ginseng and Vitis genus plant for treating and preventing neuro-degenerative disease and enhancing memory power.

2. The pharmaceutical composition according to claim 1, said ginseng comprises a root, leaf, fruit, or rhizome of a processed ginseng or a wild ginseng.

3. The pharmaceutical composition according to claim 2, said processed ginseng is a processed ginseng which is treated with heating at the temperature ranging from 70 to 200° C. for the period ranging from 0.5 to 20 hours so as to make a ratio of ginsenoside (Rg3+Rg5+Rk1) to (Rb1+Rb2+Rc+Rd) of over 1.0.

4. The pharmaceutical composition according to claim 1, said ginseng is selected from Panax ginseng, Panax quinquefolia, Panax notoginseng, Panax japonica, Panax trifolia, Panax pseudoginseng, Panax vietnamensis, Panax elegatior, Panax wangianus or Panax bipinratifidus.

5. The pharmaceutical composition according to claim 1, said “Vitis genus plant” is selected from Vitis vinifera, Vitis amurensis, Vitis flexuosa, Vitis coignetiae, Vitis thunbergii, Vitis riparia, or Vitis kaempferi.

6. The pharmaceutical composition according to claim 1, said Vitis genus plant comprises a fruit, seed, root, leaf or stem of Vitis genus plant.

7. The pharmaceutical composition according to claim 1, said extract is soluble in water, C₁-C₄ lower alcohol, or the mixtures thereof.

8. The pharmaceutical composition according to claim 1, said extract of combined herb is an extract of combined herbs consisting of ginseng and Vitis genus plant with the mixed ratio ranging from 1:0.01-1000 (v/v).

9. The pharmaceutical composition according to claim 1, said neuro-degenerative disease” is selected from stroke, Alzheimer type dementia, cerebrovascular type dementia, Huntington's disease, Pick's disease, Creutzfeldt-jakob's disease, dementia caused by cephalic damage, or Parkinson's disease.

10. (canceled)

11. A method for treating neuro-degenerative disease in a mammal or animal suffering from said disease comprising administering an effective amount of an extract of combined herbs consisting of ginseng and Vitis genus plant, together with a pharmaceutically acceptable carrier to said mammal or animal in need thereof

12. A health functional food comprising an extract of combined herbs consisting of ginseng and Vitis genus plant as an active ingredient for alleviating or preventing neuro-degenerative disease and enhancing memory power.

13. The health functional food according to claim 1, said health functional food is provided as powder, granule, tablet, capsule or beverage type.

14. A health care food comprising an extract of combined herbs consisting of ginseng and Vitis genus plant for alleviating or preventing neuro-degenerative disease and enhancing memory power, together with a sitologically acceptable additive.

15. (canceled)

16. A method for preparing extract of combined herbs consisting of ginseng and Vitis genus plant comprising the steps of; drying the fruit, seed, root, leaf or stem of ginseng and Vitis genus plant and heating the ginseng with high temperature ranging from 70 to 200° C. for the period ranging from 0.5 to 20 hours so as to make a ratio of ginsenoside (Rg3+Rg5+Rk1) to (Rb1+Rb2+Rc+Rd) of over 1.0 at 1^{st} step; mixing the plant materials with 1 to 50-fold weight (w/v) of water, C₁-C₄ lower alcohol or the mixture thereof and extracting the solution by reflux extraction, cold water extraction, ultra-sonication or other conventional extraction at the temperature ranging from 10 to 150° C. for the period ranging from 0.5 to 20 hours; filtering the residue or precipitating the suspended solution prepared by adding water with cold ethanol to afford their supernatant at 2^{nd} step; drying the filtrate or the supernatant at the temperature ranging from 40 to 80° C.; mixing each dried extract with the mixed ratio based on the dried weight of each herb (w/w) ranging from 1:0.01-1000.
